(19) United States
(12) Patent Application Publication
(10) Pub. No.: US 2018/0280071 A1
(43) Pub. Date: Oct. 4, 2018

(54) Generator for Supplying a Coagulation Instrument and Control Method for Same

(71) Applicant: Erbe Elektromedizin GmbH, Tuebingen (DE)

(72) Inventors: Bernhard Tobias Nold, Tuebingen (DE); Alexander Neugebauer, Moessingen (DE); Michael Ederer, Stuttgart (DE); Klaus Fischer, Nagold (DE); Markus Enderle, Tuebingen (DE); Jay Wagenpfeil, Stuttgart (DE); Julia Rex, Stuttgart (DE); Ronny Feuer, Esslingen (DE); Oliver Sawodny, Stuttgart (DE)

(21) Appl. No.: 15/938,876

(22) Filed: Mar. 28, 2018

(30) Foreign Application Priority Data
Mar. 29, 2017 (DE) 10 2017 106 747.7

(51) Int. Cl. A61B 18/12

(52) U.S. Cl. CPC A61B 18/1206; A61B 2018/00589

(57) ABSTRACT

The device and method according to the invention allow a particularly fast, gentle, and secure fusion of vessels between two coagulation electrodes. Resistance oscillations are generated in the biological tissue, said oscillations alternatingly moving above and below a tissue resistance value of, e.g., 50 Ohm. Subsequently, a phase of slowed tissue cooling is passed, during which phase a current is applied to the tissue with chronologically decreasing coagulation voltage to achieve a substantially slowed cooling process compared to the instant voltage disconnection. In doing so, a good fusion of the collagen of the pressed-together vessel walls and a mechanically stable solidification of the collagen is achieved. Due to the mentioned process control, the required fusion time is shortened compared to conventional methods, the unwanted damage to surrounding tissue is reduced, and the closure of the vessel is more secure.

### CROSS-REFERENCE TO RELATED APPLICATIONS

This application claims the benefit of German Patent Application No. 10 2017 106 747.7 filed Mar. 29, 2017, the contents of which is incorporated herein by reference as if fully rewritten herein.

### TECHNICAL FIELD

The invention relates to a generator for supplying a tissue fusion instrument, in particular an instrument for vessel fusion. At the same time, the invention relates to a method for the control of a generator for supplying such an instrument.

### BACKGROUND

The surgical application of coagulation instruments, in particular tissue fusion instruments, on the patient occurs under considerable pressure of time in most cases. If many coagulation measures are to be performed during a surgical intervention, in particular the closure of many vessels and, optionally, the severing of said vessels, it is important that the vessels be closed within the least possible time. The minimum possible damage and coagulation of the surrounding tissue is to occur in order to minimize undesirable lesions. On the other hand, the closure must be secure so that closed and subsequently severed vessels will not open during or after surgery and thus result in bleeding.

Typically, vessel fusion takes place between two branches of a fusion instrument to which a high-frequency coagulation is applied and which compress a vessel grasped between them and which heats it by current flow-through.

Such an instrument and associate coagulation processes can be inferred from publication U.S. Pat. No. 8,216,223 B2. The instrument is associated with a system that generates a test pulse to the instrument at the start of a coagulation process in order to detect the tissue impedance. Additionally or alternatively, the system may determine characteristics of the electrosurgical instrument at the start of the treatment. Then, the system determines whether a reaction of the tissue is to be recorded and determines a desired impedance trajectory based thereon. Considering this, the system determines the impedance of its target value based on a desired rate of change. Then the system monitors the maintenance of this desired impedance trajectory, in which case it detects the temperature, the tissue type or the like. In addition, the system is able to detect the quantity of energy delivered to the tissue during the sealing process and to stop the continued delivery of energy for a specified period of time when the impedance exceeds a threshold above the value of the initial impedance. When the application of energy to the tissue is completed, the system may provide a cooling period. The cooling period acts to solidify the collagen in the sealed tissue, in which case the cooling time is a fixed period of time or an adaptive period of time that is a function of values that are related to the tissue fusion process. Upon expiration of the cooling period, the sealing process is completed. The system may comprise active cooling elements to accelerate cooling such as, for example, heat pipes or Peltier elements.

Publication U.S. Pat. No. 5,827,271 also illustrates a tissue fusion instrument, in which the vessels are compressed between two energized branches and through which current flows and which can thus be heated in order to fuse the tissue or the vessel. After fusion has taken place, the power output to the instrument is lowered to a very low level in order to achieve cooling of the tissue within the fastest possible time. Alternatively, a very low power of 1 Watt may be output to the tissue in order to continue to keep the electric circuit closed via the tissue. Such a low power output does not delay the cooling process.

### SUMMARY

The most homogeneous possible structure is to be achieved in the coagulated tissue. The achievement of a homogeneous tissue structure and a simultaneously short sealing period are in a target conflict. It is the object of the invention to state a concept with which the homogeneity and thus the reliability of the seal can be improved and the sealing time can be shortened.

The generator according to the invention generates a coagulation voltage for operating the instrument for tissue fusion in several stages. To do so, the generator is disposed to first heat the tissue to the boiling temperature of the tissue fluid during a first stage, so that steam may form. As soon as the tissue has been sufficiently heated, the device moves into a second stage according to a first aspect of the invention. In it, the oscillation of the tissue resistance is triggered. In this phase, the tissue resistance assumes alternatingly high and low values. After high tissue resistances with relatively dry tissue or steam bubbles in the tissue and low tissue resistance with moist tissue or tissue without steam bubbles have been associated, it is not only the tissue resistance that oscillates but the tissue condition also pulses, thus potentially resulting in a pulsing stress of the tissue. Furthermore, by periodically reducing the electrical energy input into the tissue, it is possible to achieve a moisture increase, steam elimination or steam liquefaction (e.g., due to condensation or escape of steam). Consequently, it is possible to effect a lowering of the resistance of the tissue and thus, effectively and/or in total, achieve an increased total energy input. The periodic reduction of the tissue resistance effects an increased current flow between the electrodes—compared to coagulation processes, wherein the operation is performed at constant high tissue resistance.

For oscillating control of the tissue resistance, a nominal resistance curve can be determined, wherein the function block comprises a regulator for the tissue resistance. The regulator measures the tissue resistance—continuously or within a narrow time grid—and compares this tissue resistance with the tissue resistance currently specified by the nominal tissue resistance curve. Based on the resultant deviation, the regulator determines the voltage to be applied to the tissue and delivers said voltage to the coagulation electrodes which thus receive a high-frequency coagulation voltage that is amplitude-modulated at a low frequency. Its value oscillates at a few Hertz, preferably less than 30 Hz (or less than 20 Hz), further, preferably less than 20 Hz or also less than 10 Hz. In many cases, a good effect is achieved with an oscillation frequency between 10 Hz and 20 Hz. The oscillation frequency may be specified in a fixed manner by an appropriate function block or be specified in a variable manner by the function block—in particular also as a function of the generator setting, of the grasped tissue and, in particular, as a function of the instrument. Consequently, the oscillation frequency can assume different values adapted to the situation at hand.

Preferably, the regulator comprises an output voltage limiting device that sets a maximum voltage and/or a minimum voltage during the oscillation of the tissue resistance. Preferably, the maximum voltage is set in such a manner that a spark formation during phases of high tissue resistance or an otherwise thermal damage to the tissue to be coagulated, as well as to the surrounding tissue, does not occur. For example, the maximum voltage may be set at a value between 80 V and 150 V, preferably 90 V to 120 V. Deviating values are possible. The minimum voltage is preferably set at a value different from zero. In this manner, a cooling of the tissue that is too fast and a condensation of the steam in the tissue that is too fast can be prevented. For example, the minimum voltage is within a range of 20 V to 40 V.

Preferably, the function block is disposed to end the resistance oscillation at a tissue resistance that is too high in order to then transition into a controlled cooling process during a third stage, said cooling process representing a second aspect of the invention. During the controlled cooling process, a current is continuously applied to the tissue at a voltage whose amplitude decreases over time, as a result of which the cooling of the tissue that would otherwise processed vary rapidly is clearly slowed. Due to the continuous application of current to the tissue grasped between the electrodes, cooling of the tissue is clearly slowed compared with the intermediate cooling to be observed during oscillation, as a result of which the temperature gradient prevailing in the tissue is decreased. Zones that pass through temperatures that are optimal for protein chaining during the cooling process are increased as a consequence of the decreased temperature gradient in the tissue. As a result of this, the involved proteins, in particular collagens, are given more time and more space for forming mechanically durable, optionally also fibrous, structures. Proteins—in particular collagen—of opposing tissue walls that are pressed against each other are able to fuse together.

Although the slowed cooling process results in a longer cooling period, the sum of the time required for the coagulation with resistance oscillation and controlled cooling is less than would be required with a coagulation without resistance oscillation and with uncontrolled cooling. Thus, an abbreviation of the time for coagulation is possible, so that a total of less than 3 sec, in particular less than 2 sec, of treatment time—including the cooling process—can be achieved. Although the time from closing the branches of the instrument to reopening same is so short, secure sealing of the vessel and thus a high-quality surgical outcome can still be achieved.

In order to determine the procedure status and, in particular, to determine the starting and end points of the different stages, a function block can monitor at least one tissue property. Such a tissue property may be the voltage applied to the tissue, the current flowing through the tissue, the applied power, the amount of energy transmitted to the tissue, the tissue resistance or the like. For example, during the first stage at the start of the application of current, the tissue resistance typically experience a phase of decrease, whereupon it passes through a minimum and then increases again. The re-increase is connected to the formation of steam in or on the tissue. According to a first aspect of the invention now, during the second stage of the coagulation process, oscillations of the tissue resistance are generated. This can be accomplished, e.g., by specifying a setpoint curve for the tissue resistance. The coagulation voltage applied to the tissue is dimensioned by means of a regulator in such a manner that the tissue resistance behaves approximately in accordance with the specified setpoint curve. If the oscillations have occurred over a specified time (for example, approximately 0.5 sec) or if a certain number of oscillations (for example, 5 to 6, preferably 7 to 10, preferably 8) have been registered, the third stage may be initiated, said stage representing a slowed cooling process.

The resistance oscillations and/or the slowed cooling allow a fusion of blood vessels with increased safety. The resistance oscillations result in a pulsed energy input into the tissue with periodic remoistening of the tissue due to condensation of the insulating steam and therefore to increased energy input. An intimate connection of the oppositely located, pressed-together tissue walls is prepared. The delayed, i.e., slowed cooling process then provides a good recombination and protein chain formation of the involved proteins, in particular the collagen. The slowed cooling process provides larger zones in the biological tissue in which—during the cooling phase—optimal temperature conditions for the formation of long, interlinked protein chains are obtained.

Further details of advantageous embodiments of the inventive generator as well as of the inventive control method can be inferred from the description, as well as from the dependent claims and the appended drawings. They show in:

### BRIEF DESCRIPTION OF THE DRAWINGS

FIG. 1 a schematized basic diagram of the generator, a connected instrument and a vessel to be fused,

FIG. 2 a schematized sectional view of a vessel grasped between two branches for coagulation,

FIG. 3 a schematized, detail of the representation of a function block,

FIG. 4 the chronological behavior of the voltage of the coagulation voltage output by the generator,

FIG. 5 the chronological behavior of the tissue resistance in reaction to the applied coagulation voltage,

FIG. 6 the power delivered by the generator to the tissue, as well as the output energy, and

FIG. 7 the chronological behavior of the current passed by the instrument through the tissue.

### DETAILED DESCRIPTION

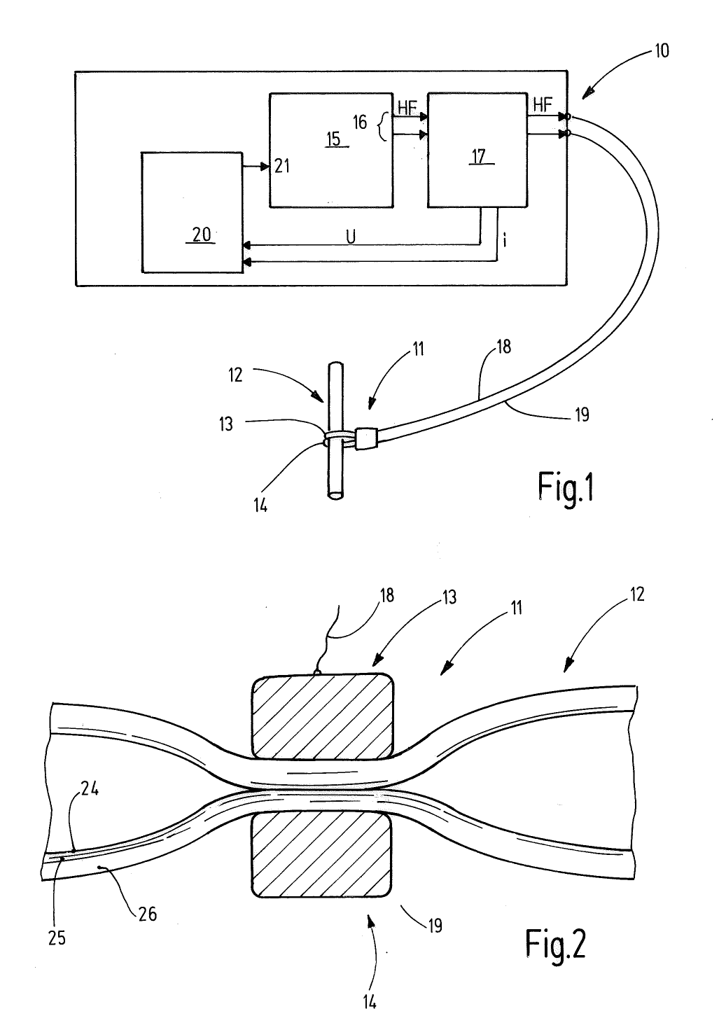

FIG. 1 shows, in a highly schematized manner, a generator 10, a tissue fusion instrument 11 supplied by it, as well as a vessel 12 that is to be closed. The instrument 11 comprises two branches 13, 14 that are disposed for grasping the blood vessel 12. The guiding and control elements such as, for example, a handle comprising an actuation lever, a short or longer shaft or the like, are not illustrated. Basically, the instrument 11 may have the design of a known tissue fusion instrument as is used for open or laparoscopic surgeries.

At least one of the branches 13, 14 is movable in order to be able to compress the tissue 12 grasped in between them as illustrated in FIG. 2, so that the insides of the tissue walls lie against each other and can be pressed against each other. Furthermore, the instrument 11 may comprise a moved, mechanical knife, an ultrasonic knife, a moved or fixed knife to which a cutting voltage is applied, or any other such type of cutting elements. The invention relates to the device 10 and, to this extent, to the application of current to the branches 13, 14 and can basically be used in any tissue or vessel fusion instrument, i.e., independent of the fact whether the instrument comprises no, one or several cutting devices for severing the fused element.

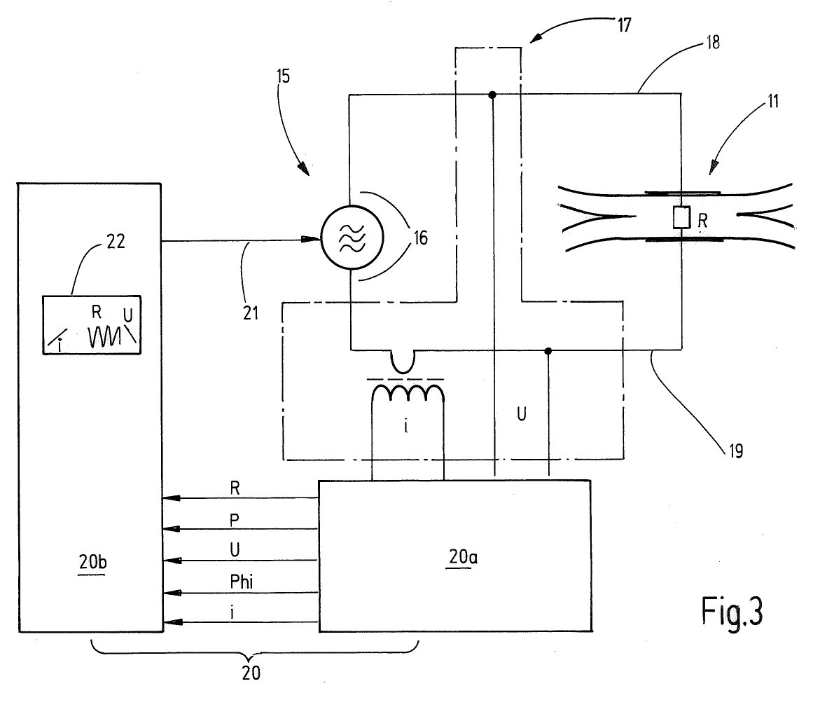

The device 10 comprises a generator 15 that provides, at an output 16, a high-frequency coagulation voltage HF that, optionally is conducted via a sensor block 17 and the lines 18, 19 to the instrument 11. The sensor block 17 is disposed to detect the intensity of the RF voltage delivered by the generator 15 and/or of the RF current delivered by the RF generator and deliver these as signals u and/or i to a function block 20 or to several function blocks 20a, 20b (see FIGS. 1 and 3) for the control of the generator 15.

The generator 15 has an input 21 via which the generator 15 is able to receive control signals. These may be analogous or digital signals that specify the intensity of the coagulation voltage u output by the generator 15. The control signals may be delivered by a function block 20 that is connected to the sensor block 17, so that the latter receives signals output by said function block. The signals may for example be signals that characterize the RF current delivered by the generator 15 and/or the RF voltage provided by the generator 15. The function block 20 may be divided into two or more function blocks 20a, 20b.

The function block 20 or the function blocks 20a, 20b may be configured as separate building blocks or as program component(s) of a generator control program or be made of any suitable other means with which the operation of the generator 15 can be controlled. They form a regulator for a parameter that is to be regulated in a stage-regulated manner; this may be, for example, the current i (e.g., in stage A), the tissue resistance R (e.g., in stage B), the coagulation voltage u (e.g., in stage C), or the power P (e.g., also in stage C). The regulator may be set to control the generator 15 during at least one of the stages within voltage limits that are fixed, chronologically constant or follow the desired progression over time. Additionally or alternatively, the regulator may be set to control the generator 15 during at least one of the stages within the current limits, resistance limits or output limits that are fixed, chronologically constant or follow a nominal progression over time. The desired progressions over time may be increasing or decreasing ramps or other regular or irregular periodic or non-periodic functions.

For a better illustration of the structure and the functionality of the function block 20, reference is made to the exemplary embodiment according to FIG. 3. The first function block 20a connected to the sensor block 17 detects the coagulation voltage u, as delivered by the generator 15 and applied to the branches 18, 19, and the current i flowing through the vessel 12 or other tissue. The intensity of the current i depends on the intensity or the applied coagulation voltage u and the intensity of degree of the current resistance R, the value of said resistance changing during the coagulation of the vessel 12 or other tissue. Based on the measured current i and the coagulation voltage u, the function block 20a can calculate, as needed (at least approximate), the existing tissue resistance R=(u/i)*cos(Phi) and/or the power P=u*i*cos(Phi) and/or the phase shift Phi between the voltage u and the current i, and output it to the function block 20b. Furthermore, the function block 20a can output the detected coagulation voltage u and/or the detected current i and/or the values calculated therefrom to the function block 20b.

The function block 20b receives—depending on the operating mode or stage of the coagulation process—at least one of the signals that characterize the tissue resistance R, the power P output to the tissue, the phase shift Phi between the coagulation voltage u and the current i, the coagulation voltage u and/or the current i that flows through the tissue. The function block part 20b of the function block 20 controls the coagulation process in that it specifies in each stage a setpoint curve for at least one of the parameters R, P, Phi, u, i, said curve being accessible, for example in a memory 22. The setpoint curve may contain several sections, each being applicable to one (or more of the parameters R, P, u, i and specifying the respective setpoint value for this parameter. Depending on the type of connected instrument 11 or depending on the adjustment at a user interface of the device 10, the setpoint(s) may vary.

Furthermore, the function block 20b comprises a regulating block that determines the difference between the respective parameter (R, P, Phi, u or i) specified by the setpoint curve and the actual value of the respectively controlled parameter R, P, Phi, u or i determined by the function block 20a. Based on this setpoint/actual difference, a setpoint voltage is derived within the function block 20b by the regulating block and output to the generator 15.

The control blocks 20a, 20b may be part of a program processed by the controller, said program functioning as follows and controlling the generator 15 as follows:

The generator 15 is capable of generating a high-frequency coagulation voltage within the range of several 100 kHz, for example 350 kHz. The voltage generated by the generator 15 may be, for example, within the range of 0 to 150 V. Other generator voltages can be used. However, in any event the voltages are such that a spark formation between the branches 13, 14 and the biological tissue, for example a blood vessel 12, does not occur. Furthermore, the generator 15 is preferably configured in such a manner that it can provide an output of up to 120 W, for example, or more. Furthermore, it is configured in such a manner that it can deliver an RF current of 2 A (or higher). With these parameters, the generator 15 is basically suitable for the fusion of the blood vessel 12 or another tissue, i.e., for the permanent closure of said vessel or tissue by means of the instrument 11.

The blood vessel 12 has a tissue endothelium 24 that forms the inner lining, i.e., the Tunika interna, of the blood vessel 12. The tissue endothelium consists of endothelial cells that form a single-layer plate epithelium, elastic fibers and connective tissue. This is seated on a middle layer 25—also referred to as the Tunika media—that consists of muscle cells, collagen fibers, elastic fibers and connective tissue. The outer layer 26—also referred to as the Tunika externa—consists mainly of connective tissue and elastic fibers. In particular the Tunika media and the Tunika externa contain collagen fibers that are to be fused together during tissue fusion.

In order to perform the tissue fusion procedure, the vessel 12 is first grasped between the branches 13, 14 and compressed in accordance with FIG. 2, so that the oppositely located inside surfaces of the tissue walls will be in contact with each other, the blood is squeezed out between the branches 13, 14, and the vessel 12 is completely clamped closed. In doing so, the branches 13, 14 apply a compressive pressure to the vessel 12.

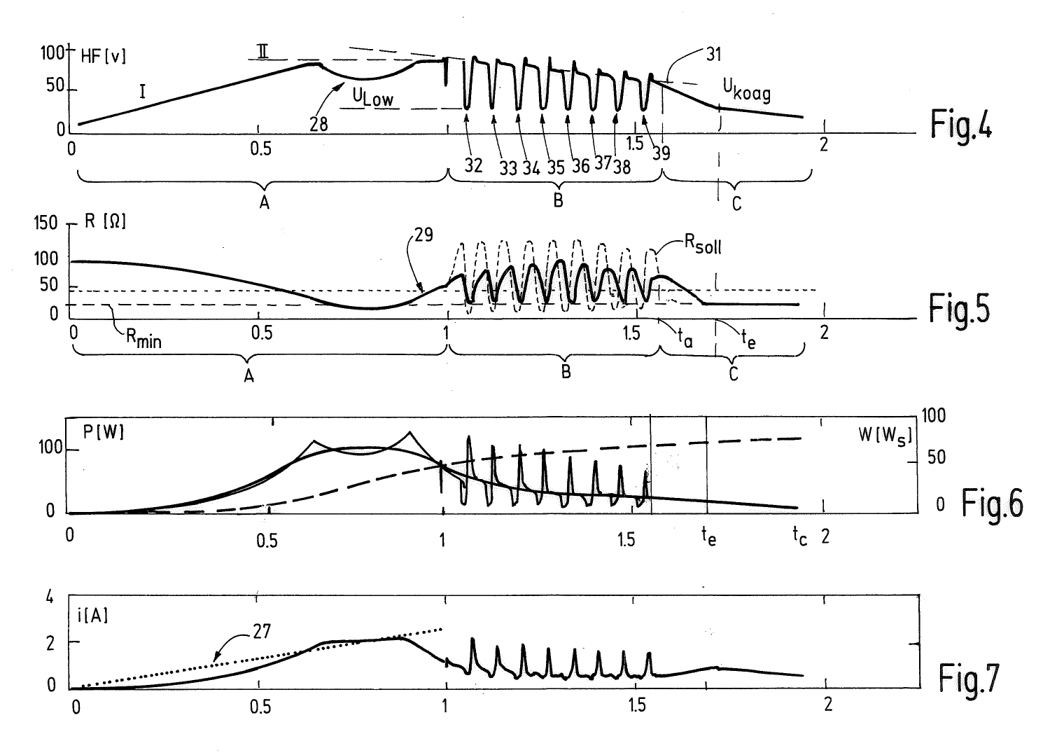

According to FIGS. 4 to 7, the fusion process begins with a first stage that, preferably, lasts at most approximately 1 sec; during this stage the vessel 12 between the branches 13, 14 is heated by the through-flowing current. Stage A can be limited to a fixed period, e.g., 2.5 sec, by means of software. For example, in stage A of the current i to be conducted through the tissue is specified as a ramp, e.g., chronologically increasing—as illustrated by a dotted line 27 in FIG. 7. In this case, the function block 20a detects the current i and delivers it to the function block 20b that acts as the regulator. The function block 20b generates a setpoint value for the generator 15 and outputs it to the generator's input 21. At the same time however, the function block 20b can also take into account the coagulation voltage HF applied to the branches 13, 14 and, for example, limit it based on a time-dependent function, e.g., a voltage ramp I (FIG. 4), for example in order to prevent damaging effects on the tissue. Limiting can be limited, e.g., initially in accordance with a specified time-dependent function, for example a linear ramp I as shown, for example, in FIG. 4, and optionally also be limited by a maximum voltage II. Due to the voltage limitation, the current i remains for a certain time (e.g., approximately 0.6 sec) below the setpoint curve 27.

While the current increase occurs, the tissue resistance R shown in FIG. 5 decreases with progressing heating of the tissue due to released tissue fluid and increasing mobility of the dissolved ions. While the resistance decreases, the current increases, so that the power regulator decreases the voltage. It falls below the limit, so that the current i now follows the specified value 27.

As an alternative to this type of control, it is also possible for the coagulation voltage u to follow a specified time-based function, e.g., as a ramp, in stages or the like. This can be specified by the function block 20b via the chronologically appropriately occurring setpoint voltage.

During the first stage, the tissue resistance R decreases—as the current increases and heating increases—initially to a value Rₘᵢₙ—that, typically, is below 50 Ohm. Due to the increasing tissue temperature, the ohmic resistance R of the biological tissue decreases to very low values such as, for example, hardly more than 20 Ohm, in many cases even below 10, 5 or 2 Ohm. As heating of the tissue increases and steam formation sets in, the tissue resistance R increases again, as can be seen at point 29 of FIG. 5. This point in time is reached after approximately 1 sec. During this process the power transmitted to the tissue is relatively high, as is shown by FIG. 6. If the tissue resistance R reaches or exceeds a limiting value of, e.g., to 50 Ohm and/or a multiple of Rₘᵢₙ and/or a certain phase shift Phi between the voltage u and the current i, stage A is completed.

At this time (at approximately 1 sec) the power P transmitted to the vessel 12 has exceeded its maximum and decreases due to the increasing resistance as a consequence of increasing steam formation and tissue desiccation, respectively. In the present exemplary embodiment, the energy W transmitted to the vessel 12 has reached approximately 50 J at this time. Alternatively, it is possible to provide other energy values.

The function block 20b can be configured in such a manner that it determines the method status in view of elapsed time, alternatively in view of the electrical energy W transmitted to the vessel 12, alternatively in view of the size and/or chronological progression of the tissue resistance R, or further alternatively, in view of the size and/or the chronological progression of the current i. A characteristic value for the method status is the beginning boiling of the tissue fluid which is accompanied by the fact that at least parts of the vessel 12 have reached boiling temperature. If the function block 20b monitors the current, this may be detected by the function block 20b in view of the current curve 27. If the function block 20b monitors the energy W transmitted to the vessel 12, the function block 20b can detect the beginning boiling of the tissue fluid by reaching a certain amount of energy (for example Watt seconds) transmitted to the vessel 12. If the function block 20 monitors the tissue resistance R, said function block is able to detect the beginning boiling of the tissue fluid by exceeding a resistance limit of 42.5 Ohm, for example, after passing through a resistance minimum.

Independently of the fact which of the said parameters is monitored by the function block 20, 20b, the latter detects—as the functions status—the end of stage A (e.g., by the beginning boiling of tissue fluid in view of the resistance R) and now guides the generator 15 into a tissue fusion phase that, preferably, lasts for half a second or minimally longer. During this phase, the generator 15 can be controlled by the function block 20b in such a manner that it generates the coagulation voltage u and the tissue resistance R with a chronological setpoint value progressing in accordance with a specified function 32 and periodic voltage decreases or voltage dips 32 to 39. The specified maximum value of the coagulation voltage u may be chronologically constant or, as inferred by FIG. 4, follow a descending, chronologically changing function, e.g., a downward sloping straight line. Other voltage progressions, e.g., in the form of a descending e-function or also an ascending voltage progression can be used.

In stage B the function block 20b preferably acts as the regulator for the tissue resistance R. To do so, the memory 22 specifies a setpoint time function for the setpoint tissue resistance R₅₀₁₁ and delivers this function to the regulator block. For example, this setpoint time function is a periodic, time-dependent function, e.g., a sine function that is shown in a dashed line in FIG. 5. The function block 20a determines the actual tissue resistance Rᵢₛₜ and also delivers it to the regulator block. It controls the generator 15 accordingly, taking into account the voltage limit II that still limits the voltage that can be maximally generated by the generator 15 to a maximum value, e.g., 90 V to 120 V. Furthermore, the function block 20b limits either the coagulation voltage u downward so that it does not go below a minimum value of e.g., 25 V. With an upward voltage limitation, it is possible to avoid spark-over on the tissue or other thermal tissue damage. The downward voltage limit A avoids a condensation of steam that is too fast or excessive.

As a result of the regulation of the tissue resistance, voltage dips 32 to 39 occur in accordance with FIG. 4, in which case the generator 15 briefly reduces its output in each instance, so that the output voltage dips from a value of approximately 90 V to 120 V to a minimum value of 20 V or 25 V, for example. Whenever there is a voltage dip, the power transmitted to the vessel 12 initially also decreases, as shown by FIG. 6. Due to the simultaneously decreasing tissue resistance, increased power is transmitted to the tissue with the periodic, respectively subsequent voltage increase. However, in the mean this power transmitted to the tissue is greater during the periodic decrease of the tissue resistance than if the coagulation were to occur completely at a high coagulation voltage u of, e.g., 100 V, and thus constant, high tissue resistance.

As a result of the periodic resistance modulation, already generated steam and/or tissue of the vessel 12 can remoisten again. There result pressure pulsations consistent with resistance oscillations illustrated by FIG. 5 and subsequent output peaks according to FIG. 6, whereby said pressure pulsations can contribute to a penetration of the vessel endothelium 24. In doing so, the protein components of the Tunika media 25 and optionally also the Tunika externa 26 of oppositely located vessel walls can come into contact with each other and fuse together.

The function block 20 can specify the method status of the vessel 12, for example in view of a time curve for the progression of the tissue resistance with a firmly or variably specified number of oscillations of the tissue resistance R or the voltage dips 32 to 39. Alternatively, it is also possible to specify the oscillations with amplitude and frequency and to detect the number of voltage dips 32 to 39 or the resistance oscillations. To accomplish this, the performed voltage decreases can be counted and, when a limit of 8 or 9, for example, is reached, stage B can be completed. In any event, stage B ends at a high tissue resistance and thus also at a high (not reduced) coagulation voltage u. Similarly, in a further modified embodiment, the function block 20 can monitor and count the output maxima and/or output minima according to FIG. 6 or the current peaks according to FIG. 7 in order to detect the method status and the end of stage B.

If the function block 20 determines in any of the previously described ways that the second stage B is completed, the function block 20 changes the actuation of the generator 15 in such a manner that it enters a controlled tissue cooling phase, stage C. In it, the vessel 12 is continued to be supplied with current in order to slow tissue cooling in a targeted manner. Consequently, according to FIG. 5, the tissue resistance R in the tissue decreases less steeply in this phase beginning at a time tₐ than during the coagulation phase during the voltage dips 32 to 39. This is accomplished in that, for example, the coagulation voltage u is guided as specified by a chronological voltage curve that is stored in the memory 22. The function block 20b specifies the coagulation voltage according to a time function, e.g., as a descending ramp. The specified voltage can be output as a control signal directly to the generator or, alternatively, to the regulator block that, on the other hand, receives the coagulation voltage u actually generated by the generator 15 and controls the generator in view of the formed difference.

The coagulation voltage u is decreased at a specified rate of −200 V/sec, for example. It is also possible to use other decreasing rates (for example −150 V/sec or −250 V/sec. Furthermore, the decreasing rate may be varied during the cooling phase, if desired.

During the cooling phase a remoistening of the tissue occurs, in which case the tissue moves—in zones—in a cooling manner through different temperature ranges of approximately 150° C. to 170° C. This is accompanied by a decrease of the tissue resistance R, which, however, due to the contused application of current, is clearly slower than the resistance decreases during the resistance oscillations in stage B. As a result of this, the temperature gradient in the biological tissue is minimized by the slowed cooling—compared with a non-slowed cooling. Zones having a relatively large volume are formed with extended duration of existence, their temperature being located in a temperature window that is favorable for protein chaining. Consequently, there is more time available for the formation of mechanically durable protein structures for each point of the involved tissue.

With the increasing elimination or evaporation of the steam, the tissue resistance R decreases to below a limiting value. This limiting value may be a specified limiting value or, alternatively, a limiting value that results from the tissue resistance during the resistance oscillations or from the resistance progression from stage A. For example, because of the minimum voltage of the generator 15, below which it is not possible to drop, the tissue resistance in stage B does not decrease as far as specified by the resistance setpoint curve. However, the occurring minimum tissue resistance Rₘᵢₙ can be registered. When the tissue resistance R reaches the registered minimum tissue resistance Rₘᵢₙ or a fixed multiple thereof (e.g., 1.5*Rₘᵢₙ), this can be used as the event for ending the voltage-guided cooling phase of stage C. At this time tₑ the function block 20 switches to output regulation. The tissue is now supplied with such a coagulation voltage u that the power P, as shown by FIG. 6, continues to decrease, for example, linearly or also consistent with a specified other curve. The end of the coagulation and thus the switching-off of the generator 15 is then initiated by the function block 20 or 20b, for example, time-controlled and/or after reaching a specific quantity of energy W and/or when reaching a specific power or based on other criteria at a time t_{c}.

Alternatively, the limiting value of the resistance may also be that value Rₘᵢₙ which is reached by the tissue resistance as the minimum before its increase 28 and/or that value to which the tissue resistance falls during the voltage dips 32 to 39. The function block 20, 20b can detect this value and put it in memory in order to use it for the detection of the end of the cooling phase as limiting value.

The device 10 according to the invention and the method concept according to the invention each allow a particularly fast, gentle and secure fusion of vessels 12 between two coagulation electrodes 13, 14. In doing so, resistance oscillations are generated in the biological tissue, said oscillations alternatingly moving above and below a tissue resistance value of, e.g., 50 Ohm. Subsequently, a phase of slowed tissue cooling is passed, during which phase a current is applied to the tissue 12 with preferably chronologically decreasing coagulation voltage in order to achieve a substantially slowed cooling process compared to an instant voltage disconnection. In doing so, a good fusion of the collagen of the pressed-together vessel walls is achieved on the one hand and a mechanically stable solidification of the collagen is achieved on the other hand. Due to the mentioned process control, the required fusion time is shortened compared to conventional methods, the undesirable damage to surrounding tissue is reduced because of the shortened time of action of the high-frequency current, and the closure of the vessel is more secure.

### REFERENCE SIGNS

| | |
|---|---|
| 10 | Device |
| 11 | Instrument |
| 12 | Blood vessel |
| 13, 14 | Branches |
| 15 | Generator |
| HF | Coagulation voltage generated by the generator 15 |
| 16 | Output of the generator 15 |
| 17 | Sensor block |
| 18, 19 | Lines |
| u | Signal characterizing the coagulation voltage RF |
| i | Signal characterizing the current of the generator 15 |
| R | Tissue resistance |
| P | Power transmitted to the tissue |
| Phi | Phase angle between u and i |
| 20 | Function block/regulator |
| 21 | Input for control of the generator 15 |
| I | Ramp for voltage limitation |
| II | Voltage limit |
| 22 | Memory |
| 24 | Vessel endothelium (Tunica interna) |
| 25 | Tunica media |
| 26 | Tunica externa |
| 27 | Default for setpoint power |
| 28 | Section of decreasing voltage u |
| 29 | Re-increase of the tissue resistance |
| W | Energy transmitted to the vessel 12 |
| 32-39 | Voltage dips |
| tₐ, tₑ | Start and end of the voltage-guided cooling phase |
| t_{c} | End of current application |

1. A device (10) for supplying energy to an instrument (11) for tissue fusion, the device (10) comprising:
a generator (15) configured to generate a coagulation voltage (u) to operate the instrument (11), wherein the instrument is configured to apply the coagulation voltage (u) to a tissue (12) to be fused to heat said tissue at least to a boiling temperature of a tissue fluid of the tissue (12), wherein the generator (15) is controlled at least based on an intensity of the generated coagulation voltage (u), and
a controller circuit configured to process a function block (20) configured to control the generator (15) and configured to control at least one of a tissue resistance (R) and a coagulation voltage (u) in an oscillating manner.

2. The device according to claim 1, wherein the function block comprises a regulator for controlling the tissue resistance (R) and comprises a resistance setpoint curve that is set for oscillating control of the tissue resistance (R).

3. The device according to claim 2, wherein the regulator comprises an output voltage limiting unit that sets a maximum voltage or a minimum voltage during the oscillation of the tissue resistance.

4. The device according to claim 2, wherein the function block (20) is configured to end oscillation of the tissue resistance at a high tissue resistance.

5. A device (10) for supplying energy to an instrument (11) for tissue fusion, the device comprising:
a generator (15) configured to generate a coagulation voltage (u) for operating the instrument (11), wherein the instrument is configured to apply the coagulation voltage (u) to a tissue (12) to be fused to heat said tissue to a boiling temperature of a tissue fluid of the tissue (12), wherein the generator (15) is controlled at least based on an intensity of the generated coagulation voltage (u), and
a controller circuit configured to process a function block (20) configured to control the generator (15) to perform a tissue cooling process with a continued application of current to the instrument (11) at a coagulation voltage (u) with a chronologically decreasing amplitude.

6. The device according to claim 5, wherein the function block (20) is configured to reduce—continuously or in several stages—the coagulation voltage (u) during the tissue cooling process, starting with the coagulation voltage (u).

7. The device according to claim 5, wherein the function block (20) is configured to reduce the coagulation voltage (u) consistent with a specified curve.

8. The device according to claim 5, wherein the function block (20) is configured to be controlled based on an intensity of a generated power (P) and is configured to monitor a status of the tissue cooling process and to transition, based on the status of the tissue cooling process, from the application of current with the decreasing coagulation voltage (u) to an application of current with a regulated power (P).

9. The device according to claim 5, wherein the function block (20) is configured to control at least one of a tissue resistance (R) and a coagulation voltage (u) in an oscillating manner.

10. A control method for a device (10) for supplying energy to an instrument (11) for tissue fusion, comprising:
supplying a coagulation voltage (u) during a coagulation phase which, if said coagulation voltage is applied via the instrument (11) to a biological tissue (12), causes a current that causes the biological tissue (12) to form steam, and
modulating the coagulation voltage (u) such that a tissue resistance (R) oscillates.

11. The control method according to claim 10, wherein the modulation of the coagulation voltage (u) is consistent with a resistance setpoint curve.

12. The control method according to claim 10, wherein the coagulation voltage (u) varies between a high value and a low value during modulation and is ended at the high value.

13. A control method for a device (10) for supplying energy to an instrument (11) for tissue fusion, comprising:
supplying a coagulation voltage (u) during a coagulation phase, which, if said coagulation voltage is applied via the instrument (11) to a biological tissue (12), causes a current that causes the biological tissue (12) to form steam, and
reducing the coagulation voltage (u) over a time (ta, tc) during a cooling phase subsequent to the coagulation phase to slow cooling of the biological tissue relative to an instant disconnection of the coagulation voltage.

14. The control method according to claim 13, further comprising:
initiating the end of the coagulation phase and the start of the cooling phase when one of a specified number of decreases of the coagulation voltage (u), the current or a tissue resistance (R) has been detected, a fixed time period has elapsed since the beginning of the coagulation phase, or a fixed amount of energy has been delivered to the biological tissue.

15. The control method according to claim 13, further comprising:
gradually decreasing the coagulation voltage (u) during the cooling phase over a given period of time by a value said coagulation voltage last had during the coagulation phase until a tissue resistance drops below a limiting value (R), and
initiating a post-heating phase during which a power (P) supplied to the instrument (11) is regulated consistent with a specified curve.
